(19) United States
(12) Patent Application Publication
(10) Pub. No.: US 2008/0138890 A1
(43) Pub. Date: Jun. 12, 2008

(54) Blood Analysis Apparatus

(75) Inventors: Yasuhiro Horiike, Ibaraki (JP); Akio Oki, Ibaraki (JP); Hiroko Kouda, Ibaraki (JP)

Correspondence Address: WENDEROTH, LIND & PONACK, L.L.P., 2033 K STREET N. W., SUITE 800, WASHINGTON, DC, 20006-1021, US

(73) Assignee: NATIONAL INSTITUTE FOR MATERIALS SCIENCE, Ibaraki (JP)

(21) Appl. No.: 11/660,162

(22) PCT Filed: Aug. 18, 2005
(86) PCT No.: PCT/JP05/15376

§ 371 Date: Aug. 6, 2007

(30) Foreign Application Priority Data
Aug. 18, 2004 (JP) 2004-238910

(51) Int. Cl. C12M 1/00

(52) U.S. Cl. 435/288.7

(57) ABSTRACT

The present invention provides a blood analysis apparatus which needs no mixing of multiple solutions described above, and has no deterioration of an analysis apparatus by application of a dry chemistry reagent which is good for storage, and thereby makes minimization of apparatus accomplishable, by introducing very small amount (several μL or less) of blood into a micro-channel which is manufactured on an insulator substrate such as a quartz plate or a polymer resin plate by the centrifugal force to conduct separation, weighing the plasma, introducing it into a flow channel of a dry chemistry reagent which has been weighed by the flow channel volume, introducing light which has the same wavelength of the color development generated by the reaction with the plasma, and measuring the attenuation degree, and a blood analysis method, which allows blood analysis of high accuracy, high reliability and cheap price. The invention provides a blood analysis apparatus which needs no special mixing and is cheap and simple by small area of a substrate, for the problems of weighing of the plasma and a substrate buffer solution and difficulties of mixing in a micro-channel which is usually under the laminar flow, deterioration of injected substrate buffer solution and area enlargement of a substrate in measurements of multiple items, in a blood analysis apparatus wherein the plasma is separated from several μL of the whole blood in a micro-channel on a substrate of about several cm, and the obtained plasma and a substrate buffer solution are mixed and test components are measured by the colorimetric method.

### TECHNICAL FIELD

The present invention relates to a blood analysis apparatus consisting of microgroove flow channels produced on a substrate made of insulating material such as quartz plate, polymer resin plate or the like, into which a sample comprising a test substance has been introduced.

### BACKGROUND ART

For diagnosis for health or diagnosis for the status of diseases in the past, a large amount of blood such as several cc was sampled from a patient, and an analysis therefor has been performed from measurement values obtained with a large-scale automated blood analyzer. Such automated blood analyzer was usually installed in medical facilities such as a hospital, and had a large scale, and needed professional qualifications for its operation.

However, in recent years, a trend for development of a new device and its practical use have increased, wherein various analysis apparatuses such as a sensor are arranged on a substrate of just several mm² to several cm² by applying a micro-processing technique which is used in manufacturing of a semiconductor device which has been in extreme advance, and body fluids such as the blood of a subject is introduced thereto, which makes it possible to understand the health state of the subject in a moment. Appearance of such cheap device allows health management of elderly people everyday at home in the forthcoming aging society, which leads to compression of health insurance benefit which is on increase. In addition, this device can be used in making quick judgment for presence or absence of infections (hepatitis, Acquired Immunodeficiency Syndrome, etc.) of a subject in the scene of the emergency treatment, which leads to suitable response. Therefore, various social effects thereof are expected, which make it as a technical field which draws unusual attention. In replacement of such automated analyzer in the past, a small, simple blood analysis method and a blood analysis apparatus have been developed for the purpose of implementing blood analysis at each home with one's own hands (e.g., see Patent Document 1).

Patent Document 1 describes a blood analysis apparatus which is made as a micromodule wherein a microgroove flow channel is installed on a polymer substrate, of which one end has a needle mounted, and blood is introduced to the groove from the needle by a pump from the other end, and introduced into a U-shaped flow channel, and centrifuged to give separation of the blood cells and the plasma, and this plasma is led to an electrochemical sensor by the pump to thereby measure the concentration of the test substance in the plasma. As results, the pH value in the blood and each concentration of oxygen, carbon dioxide, sodium, potassium, calcium, glucose, lactic acid, etc. are measured.

However, with the electrochemical sensor, it is not easy to measure the activity of each enzyme of γ-GTP, GOT and GPT, which are test substances for measurement of hepatic functions, or concentrations of total cholesterol or neutral fatty acid, and the activity of each enzyme of γ-GTP, GOT and GPT is made to be measurable with the use of the colorimeter method in Patent Document 2. Accordingly, with a large-scale automated blood analyzer, ions such as sodium and potassium are measured electrochemically, but other items are measured mainly by the colorimeter method.

[Patent Document 1] Japanese Patent Laid-Open No. 2001-258868

[Patent Document 2] Japanese Patent Application No. 2003-346436

[Patent Document 3] Japanese Patent Application No. 2003-126758

### DISCLOSURE OF THE INVENTION

With the automated blood analyzer, the blood is sampled in a large amount, and mixing of a large amount of the plasma and a large amount of the substrate buffer solution is carried out in a test tube, and the reaction is measured by absorbance. However, when this method is fulfilled with the use of a substrate comprising a polymer and the like of several cm² consisting of micro flow channels into which a very small amount of blood has been introduced, first, a special flow channel is needed for homogenous mixing of the plasma and a large amount of the substrate buffer solution as described in Patent Document 2 and Patent Document 3.

With Patent Document 2, stirring is performed by the centrifugal force with the use of two centers of the chip, and with Patent Document 3, the mixing room is prepared in the multistage. However, with the former, the system is complicated to change the two centrifugal centers, and with the latter, the stage number of the mixing room needs to be increased to achieve homogenous mixing, and a mixer is arranged for implementing the blood analysis chip, which increases the area. In addition, the mixing ratio of the plasma or the serum and the substrate buffer solution is generally in a range of 1:10 to 40 although it increases by a test substance, and, to maintain this ratio, strict weighing of the plasma and the substrate buffer solution is very important, and to fulfill this with a micro flow channel, high-precision processing is needed for the micro flow channel for storage of the plasma and the substrate buffer solution. In addition, if the substrate buffer solution is stored for a long time in the inside of the flow channel substrate comprising a polymer material, it is denatured by the oxygen penetrating the polymer material, which makes its storage very difficult. On the other hand, it may be good if the solution weighed from the outside is discharged into the micro flow channel for storage without storage of the substrate buffer solution, but it needs that a vessel for the substrate buffer solution is provided for the measurement apparatus, and the tube for discharge is positioned in the right location to the micro flow channel for storage, which cannot avoid complication and enlargement of a measurement apparatus, and which is not practical for home.

On the other hand, a minimized blood analysis apparatus are on the market such as the system of Leftron (Germany, Boehringer Mannheim), Vitros (US, Johnson & Johnson), Fuji Drychem (Fuji Photo Film Co., Ltd) and SpotChem (Arkray Inc, Kyoto), using the dry chemistry method. The structure is shown in FIG. 1 for an example of Fuji Drychem, wherein a reagent layer 102 containing a reagent which is necessary in an analysis reaction is coated on the transparent support of the reagent slide (plastic film) 101. A reagent which is necessary in the reaction is completed for preparation in this reagent layer, and held as dried in the gel. Further, a reflection layer 103 which allows reflection photometry, and a layer 104 for development which develops a sample evenly are laminated on it to form a four-layered structure. About 10 μL of a sample 105 of the plasma or the serum is dropped into the layer for development on the upper side of this film, the sample is evenly developed radially in a transverse direction by the capillary phenomenon of the layer for development, and then penetrates the reflection layer. The reflection layer and the reagent layer which is mixed with the gel absorb the blood components, which absorb and hold a constant amount of the sample per unit area. The sample which is absorbed and the reagent which is contained in the reagent layer react together, and give color development corresponding to the sample component. Certain incident light 104 corresponding to the color development is irradiated from the support side to thereby measure the intensity of the reflection light 105, which leads to estimate of the concentration of the component in the sample. Herein, 106 is a slit, and 107 is a mount made of plastic.

The dry chemistry method is an analysis method wherein if the reagent which has been kept as dried or apparently dried encounters a sample which is in the liquid state at the time of measurement, a chemical reaction proceeds in a matrix which is contained in the reagent for the first time. If the reagent is prepared in advanced, it has a big advantage of no need for strict weighing and mixing of the plasma or the serum and the substrate buffer solution in the colorimetric method described above.

Herein, the present invention has been done in refection of such circumstances, and an object of the invention is to provide a substrate structure which comprises introducing very small amount (several μL or less) of blood into a microgroove flow channel which is manufactured on an insulator substrate such as a quartz plate or a polymer resin plate, and performing centrifuge for it, introducing the subject from the plasma into a flow channel for electrochemical sensor and a flow channel which has received a dry chemistry reagent, and introducing light which has the same wavelength as that of certain color development generated by the reaction with the plasma in the flow channel for the dry chemistry reaction to receive light.

To solve the above problems, the first invention of the present application is to provide a blood analysis apparatus for a test component in the plasma by forming a micro flow channel which has various functions on a substrate, introducing a whole blood sample from an opening for introduction of blood to a flow channel for separation of the blood cells and the plasma with the use of centrifugal force to thereby obtain a plasma fraction within the substrate, delivering the plasma by a guiding flow channel, introducing it into a flow channel which has received a dry chemistry reagent to have the test component in the plasma react to the dry chemistry reagent, and introducing a certain light for measurement to thereby measure the change of permeation degree by an optical receiver.

The second invention of the present application is to provide the apparatus according to the first invention of the present application wherein a blood-collection assembly where a blood-collection needle and a tube for the whole blood reservoir are connected, is inserted into an opening for introduction of the whole blood which is formed in the substrate of the he blood analysis apparatus, and the whole blood is delivered to a flow channel for separation of the blood cells and the plasma from the blood introduction opening via the guiding flow channel for the blood, by rotating the assembly about the first rotation axis which is installed on the outside of the substrate in the axial direction of the blood-collection assembly, and further it is isolated into the blood cell fraction and the plasma fraction with the use of centrifugal force. Therefore, it requires no aspiration by a pump or pressure from the outside, and an opening and tube for guidance thereof, which leads to easy design of the apparatus, and helps minimization of the apparatus.

The third invention of the present application is to provide the apparatus according to the first or the second invention of the present application which has a structure of a flow channel for separation of the blood cells and the plasma by a flow channel which comprises plural grooves. First, an apparatus is provided wherein the whole blood introduction opening is connected with a guiding flow channel for delivery of the whole blood, the guiding flow channel for delivery of the whole blood is connected with one groove which is connected to the lower side from the view of the blood introduction opening of the groove group in which plural groove flow channels installed in an approximately arc shape about the blood introduction opening and in the direction of the blood introduction opening are formed, and the whole blood is introduced into the groove flow channel via the guiding flow channel for delivery of the whole blood by rotation about the first rotation axis, and it is rotated as itself, whereby the blood cell fraction is received in the lower side of the groove flow channel, and the plasma fraction is received in the upper side of the groove flow channel. The aforementioned groove flow channel is described in JP-A No. 2001-258868, which is characterized that the test component can be directly measured by installing an electrochemical biosensor in the plasma which is supernatant in the upper side. If this invention is applied to the present invention, it is possible to distribute the plasma which has been weighed approximately to the following channel for measurement.

The fourth invention of the present application is to provide the apparatus according to the first or the second invention of the present application wherein the flow channel for separation of the blood cells and the plasma obtains the plasma by an approximately U-shaped flow channel, specifically a flow channel in which the letter U is formed as slightly open to the outer side. The present invention provides an apparatus wherein one end of the U-shaped flow channel is connected with the guiding flow channel for delivery of the blood which is connected with the whole blood introduction opening, the whole blood is introduced into the U-shaped flow channel by rotation about the first rotation axis, which is installed on the substrate upward from the view of the blood introduction opening from the lower end of the U-shaped flow channel, and if centrifuge continues as itself, the blood cell fraction is received in the curved part on the lower end of the U-shaped flow channel, and the plasma fraction is received in the upper part thereof. The basics of the present invention have been described in Japanese Patent Application No. 2003-346436 of Patent Document 2, but the new application of the present invention by introducing the supernatant plasma into the sensor of the dry chemistry method, is described in the present application.

The fifth invention of the present application is to provide an apparatus based on the first or the third invention of the present application wherein a test component is measured by the dry chemistry method. Specifically, each of the other ends of the plural groove flow channels which is described in the third invention of the present application has a guiding flow channel for the plasma, a plasma reservoir for weighing the plasma, a capillary valve and each flow channel for introduction of a dry chemistry reagent, which are connected in a series in this order. Rotation is made about the second rotation axis described in the third invention of the present application, and by the centrifugal force, the plasma is introduced once into each of the plasma reservoirs for weighing the plasma from the plasma fraction receiving part via each of the plasma guiding flow channels. If the centrifugal force is further increased, weighed plasma is introduced into each of the flow channels for introduction of a dry chemistry reagent via each of the capillary valves, and the plasma is further is introduced by the centrifugal force from the side of the flow channel where weighed dry chemistry reagent has been introduced. The dry chemistry reagent proceeds in reaction with the test component of a constant concentration in the plasma, in the longitudinal direction of the reagent introduction channel. A series of delivery of the plasma is carried out without the use of a pump or air pressure, whereby simplicity of operation and the minimization of the apparatus are accomplished.

The sixth invention of the present application is to provide an apparatus which is based on the first or the fourth invention of the present application, wherein a laminated substrate is formed by installing plural guiding flow channels for delivery of the blood, which are branched in the flow channel of the other end of the U-shaped flow channel, and which a guiding flow channel for the plasma, a blood reservoir, a plasma reservoir for weighing, a capillary valve and a flow channel for introduction of the plasma are connected with each other in a series in this order on the same substrate, and further installing a layer for development of the plasma right below the flow channel for introduction of the plasma, and joining right below a substrate which has a region where plural holes are installed, and a flow channel for injection of a dry chemistry reagent for calorimetric measurement in this order, which are in conformity in the vertical direction. The areas are the same for each of the layer for development of the plasma, the region where plural holes are installed, and the flow channel for injection of a dry chemistry reagent for colorimetric measurement. Then, with rotation about the second rotation axis located in the outside of the substrate from the view from the flow channel in the approximately perpendicular direction to the U-shaped flow channel, the plasma in the U-shaped flow channel is introduced by the centrifugal force into the flow channel for injection of a dry chemistry reagent for colorimetric measurement.

The plasma from the plasma developing channel is introduced into the flow channel for injection of a dry chemistry reagent for colorimetric measurement through the hole at the lower part, wherein the centrifugal force used in this introduction is in the perpendicular direction to the hole. However, the end in the direction where the centrifugal force is applied to the flow channel for plasma reservoir serves as a wall, and the flow channel for plasma reservoir is full of the plasma, which leads the plasma received pressure by the centrifugal force to be introduced into the hole below. Like this, it is possible to introduce the plasma into the flow channel for dry chemistry reagent only with the use of the centrifugal force.

The seventh invention of the present application is to provide the apparatus as described in the fifth or the sixth invention of the present application wherein a reservoir for receiving extra plasma is installed on the wall of the upstream side of the plasma reservoir for weighing. If extra plasma is introduced when the determined amount of the plasma is introduced into the plasma reservoir by the centrifugal force, it is wasted into a waste plasma reservoir in the outside via a guiding flow channel for delivery of the waste plasma, which is installed in the lateral wall, whereby a correct amount of the plasma is provided into the plasma reservoir for weighing.

The eighth invention of the present application is to provide the apparatus as described in the fifth or the sixth invention of the present application which is characterized that an opening for injection and an opening for discharge are installed in a flow channel for dry chemistry reagent, in order to inject the dry chemistry reagent to the flow channel to gather a determined amount of the dry chemistry reagent, and discharge the extra reagent.

The dry chemistry reagent is prepared as a gel state, which makes it easy to introduce a certain amount of the gel into the micro flow channel according to the present invention.

The ninth invention of the present application is to provide the apparatus as described in the sixth invention of the present application wherein the flow channel for injection of a dry chemistry reagent for calorimetric measurement is characterized that the both ends in the longitudinal direction have walls installed in 45 degrees, and the inside is coated with metal and the like, and a fiber for optical introduction and emission is introduced vertically from the lower part or upper part of the substrate as to reflect the light with the wall of 45 degrees.

The inside of the flow channel is coated with a material such as metal which prevents optical leakage and intrusion, in order to introduce a certain wavelength from the lower part or the upper part of the substrate in the flow channel for calorimetric measurement, to investigate the degree of color development or coloration which is given by the dry chemistry reagent by reaction with the test component or ammonia in the introduced plasma, and to propagate it through the inside of the flow channel without attenuation by photo absorption by the inner wall, etc., to thereby measure the attenuation degree of the introduced light only by the chemical reaction of color development, coloration, etc. using a detector and to prevent the stray light from the outside from intrusion. Furthermore, the both ends of the flow channel in the longitudinal direction have walls installed in 45 degrees, and a fiber for optical introduction and emission is introduced around it, which leads that light is reflected with the wall of 45 degrees and the optical introduction and reception are carried out in high efficiency.

The tenth invention of the present application is to provide the apparatus as described in the sixth invention of the present application wherein a fiber for optical introduction and emission is introduced to the both ends of the flow channel of the longitudinal direction in conformity in both of the directions with the flow channel axes, while the inside of the flow channel for calorimetric measurement is coated with a photo-screening material such as metal in the flow channel for injection of a dry chemistry reagent for colorimetric measurement, which is the same as in the ninth invention of the present application.

The eleventh invention of the present application is to provide the apparatus as described in the sixth invention of the present application wherein the swollen amount is measured in advance when weighed certain amount of plasma is swollen by reaction with the gel containing the dry chemistry reagent, and the flow channel for injection of a dry chemistry reagent for calorimetric measurement is designed and constructed with the swollen volume.

The twelfth invention of the present application is to provide the apparatus as described in the sixth invention of the present application wherein the flow channel for injection of a dry chemistry reagent for colorimetric measurement is designed and constructed with a constant channel volume although weighed certain amount of plasma is swollen by reaction with the gel containing the dry chemistry reagent.

The gel is usually swollen to about twice to five times, but it is possible to measure the attenuation degree by introducing certain light to the total swollen volume, and the measurement values having large S/N ratio are obtained in the eleventh invention of the present application. However, reversely, it is difficult sometimes to make measurement if the attenuation degree is too high, and the area occupied by the flow channel on the substrate increased, which leads to enlargement of the substrate area. On the other hand, in the twelfth invention of the present application, it is possible to measure multiple items of the test component concentrations even with the flow channel of a constant volume, by knowing in advance the concentration of the test component of the user and preparing a measurable dry chemistry reagent, although the introduced reagent is discharged for the reagent which has swollen largely, so the attenuation degree of the measured light is small, and the S/N ratio is measured as low by the eighth invention of the present application. Both of the inventions are used in combination for the real substrate.

The thirteenth invention of the present application is to provide the apparatus as described in the sixth invention of the present application wherein in the flow channel for injection of a dry chemistry reagent for calorimetric measurement, a substrate of a flow channel of the dry chemistry reagent for generation of ammonia gas, a substrate where a number of holes are installed and a flow channel for calorimetric measurement for generation and detection of ammonia gas wherein a coloration material is introduced are laminated in this order in conformity in the vertical direction, and further wherein a flow channel for detection of other items and a flow channel for generation of ammonia gas are installed on the same substrate. In measurement of the uric acid, creatinine or urea nitrogen, it is difficult to make direct measurement, and although ammonia gas is generated after the reaction with each enzyme, the ammonia gas generated from the flow channel for generation of ammonia gas should be introduced into a flow channel into which a reagent is uniformly introduced to react to the ammonia gas to develop color, but the present invention makes this possible.

The fourteenth invention of the present application is to provide the apparatus as described in the first invention of the present application wherein an analysis for a test component by a conventional electrochemical sensor is constituted on the same lamination substrate, in addition to the analysis for a test component from the plasma with the use of the dry chemistry reagent.

In general diagnosis, measurement for Na⁺, K⁺, Ca⁺⁺, pH, etc. is essential, which is difficult to measure with the dry chemistry reagent. Therefore, the accuracy of the diagnosis increases by measuring the test component at the same time for multiple items from the collected blood.

### BRIEF DESCRIPTION OF THE DRAWINGS

FIG. 1 is a view showing the structure of the diagnosis chip with the use of conventional dry chemistry.

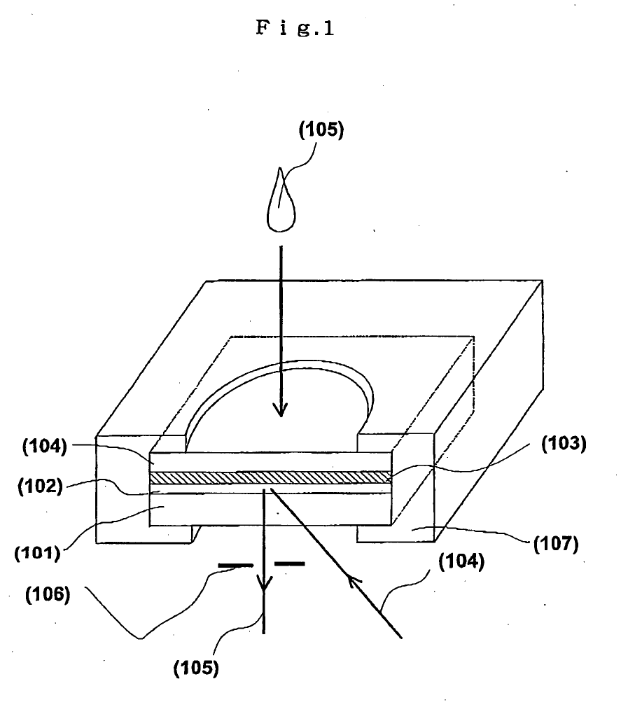

FIG. 2 is a view showing schematically the uppermost layer of the substrate among each of the lamination substrates constituting the blood analysis apparatus.

FIG. 3 is a view showing schematically the second layer of the substrate which comprises introduction of the whole blood, separation of the blood cells and the plasma, delivery of the plasma, weighing of the plasma, a capillary valve, a flow channel for introduction of a dry chemistry reagent, and a flow channel for introduction of ammonia gas-generating reagent, among each of the lamination substrates constituting the blood analysis apparatus.

FIG. 4 is a view showing schematically the third layer of the substrate which comprises multiple holes for developing ammonia gas, among each of the lamination substrates constituting the blood analysis apparatus.

FIG. 5 is a view showing schematically the fourth layer of the substrate which comprises a flow channel for introduction of a colorant reagent which reacts to ammonia gas among each of the lamination substrates constituting the blood analysis apparatus.

FIG. 6 is a view showing schematically the lowest layer of the substrate among each of the lamination substrates constituting the blood analysis apparatus.

FIG. 7 is a view showing the structure of the lamination substrates wherein a flow channel is installed for other analysis items than detection of ammonia gas.

FIG. 8 is a view showing the flow channel structure of the multiple layers comprising a flow channel for detection of ammonia gas 223.

FIG. 9 is a view showing schematically the uppermost layer of the substrate among each of the lamination substrates constituting the blood analysis apparatus wherein plural colorimetric sensors and plural electrochemical sensors are integrated on one substrate.

FIG. 10 is a view showing schematically the second layer of the substrate among each of the lamination substrates constituting the blood analysis apparatus wherein plural colorimetric sensors and plural electrochemical sensors are integrated on one substrate.

FIG. 11 is a view showing schematically the third layer of the substrate among each of the lamination substrates constituting the blood analysis apparatus wherein plural calorimetric sensors and plural electrochemical sensors are integrated on one substrate.

FIG. 12 is a view showing schematically the lowest layer of the substrate among each of the lamination substrates constituting the blood analysis apparatus wherein plural colorimetric sensors and plural electrochemical sensors are integrated on one substrate.

FIG. 13 is a view showing the section of the structure which is shown as dashed oblique line in FIG. 12 of the electrochemical sensor region.

FIG. 14 is a view showing the relation of absorbance to glucose concentration in the glucose standard solution.

FIG. 15 is a view showing the relation of absorbance to urea concentration change in the urea standard solution.

Furthermore, reference numerals will be explained below.

### REFERENCE NUMERALS

101 transparent support for reagent slide (plastic film)

102 reagent layer containing reagent necessary for analysis reaction

103 reflection layer

104 development layer

105 sample of plasma or serum

106 slit

107 plastic mount

201 uppermost layer of substrate

202 second layer of substrate

203 third layer of substrate

204 fourth layer of substrate

205 lowest layer of substrate

206 hole for connection of substrate

207 cylinder which passes through bole for connection of substrate

208 needle made of stainless tube

209 stainless tube

210 glass tube

211 guiding flow channel for delivery of blood

212 first rotation axis

213 plural blood reservoir grooves

214 second rotation axis

215 guiding groove for delivery of blood

216 plasma reservoir for weighing

217 hole for introduction of air

218 guiding groove

219 hole for introduction of air

220 guiding flow channel for extra plasma

221 waste reservoir for extra plasma

222 capillary valve

223 channel for detection of ammonia gas

224 channel for detection of other items than ammonia gas

225 opening for introduction of gel

226 opening for discharge of gel

227 declined wall of 45 degrees

228 hole for penetration

229 fiber

230 aluminum film

231 halogen lamp or tungsten lamp

232 bandpass filter

233 optical receiver

234 plural holes

235 flow channel containing colorant which reacts with ammonia gas

236 declined wall of 45 degrees

237 aluminum film

238 through-hole for fiber

239 fiber

240 hole for injection

241 hole for discharge

242 hole for insertion of glass tube

501 uppermost layer of substrate

502 second layer of substrate

503 third layer of substrate

504 lowest layer of substrate

505 through-hole for connection of substrate

506 cylinder which is inserted into through-hole

507 no-pain needle

508 stainless tube

509 glass tube

510 approximately U-shaped tube

511 first rotation axis

512 blood cell reservoir

513 column for prevention of blood cell reflux

514 second rotation axis

515 plasma reservoir

516 guiding flow channel for delivery of plasma

517 capillary valve

518 plasma reservoir for weighing of plasma

519 guiding flow channel for introduction of plasma

520 guiding flow channel for delivery of waste plasma

521 waste plasma reservoir

522 layer for development of plasma

523 sensor region of dry chemistry-colorimetric method

524 sensor region of electrochemical sensor method

525 opening for introduction of calibration solution

526 guiding flow channel for introduction of calibration solution

527 guiding flow channel for waste solution after calibration

528 opening for waste

529 region in where plural holes are installed

530 sensor region of dry chemistry-colorimetric method

531 sensor region of electrochemical sensor method

532 flow channel for injection of dry chemistry reagent for measurement

533 flow channel for injection of dry chemistry reagent for measurement

534 flow channel for injection of dry chemistry reagent for measurement

535 flow channel for injection of dry chemistry reagent for measurement

536 hole for injection

537 hole for discharge

538 hole for incidence of measurement light

539 hole for emission

540 fiber

541 fiber

542 electrochemical sensor region

543 Ag/AgCl electrode

544 KCl-saturated Ag/AgCl reference electrode

545 silver carbon wiring

546 outside electrode for taking signal out

547 ionophore film

548 film for prevention of KCl dissolution and loss

549 hole for insertion of glass tube

### BEST MODE FOR CARRYING OUT THE INVENTION

### First Embodiment

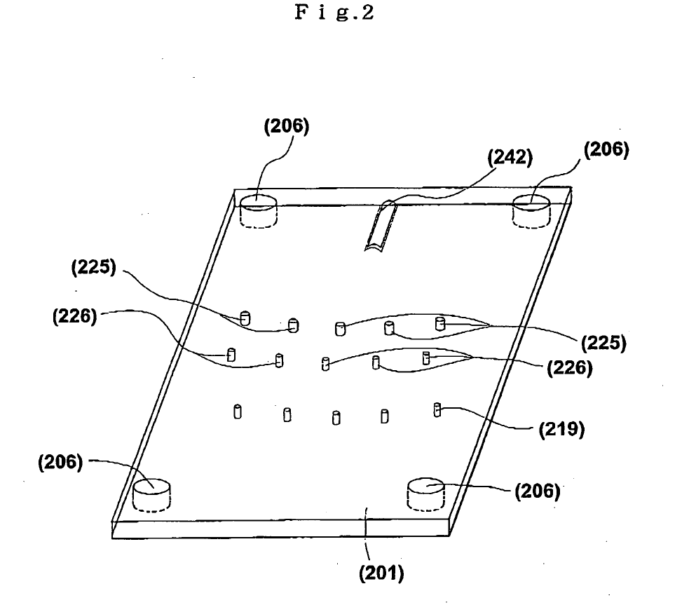

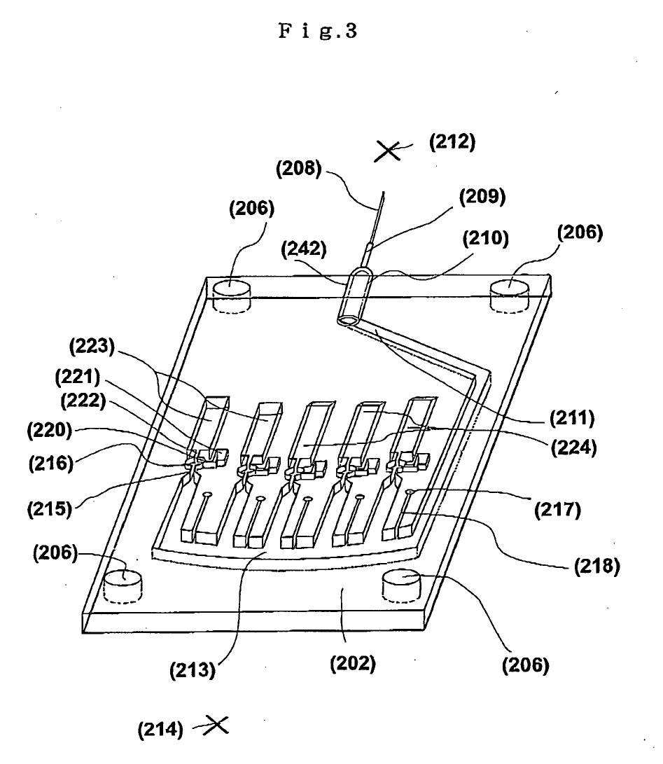

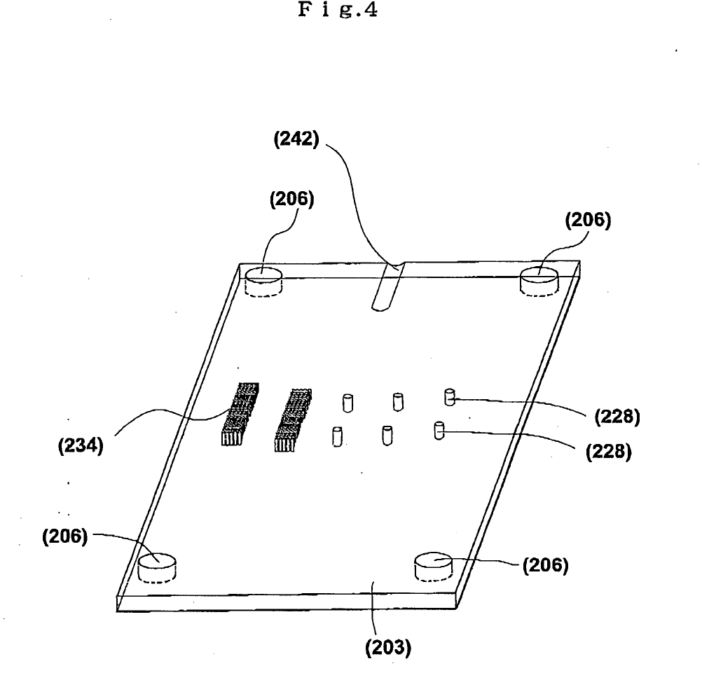

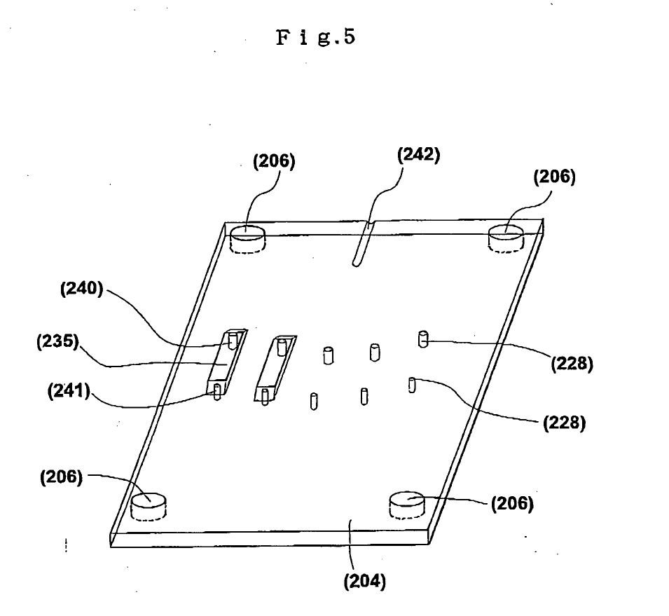

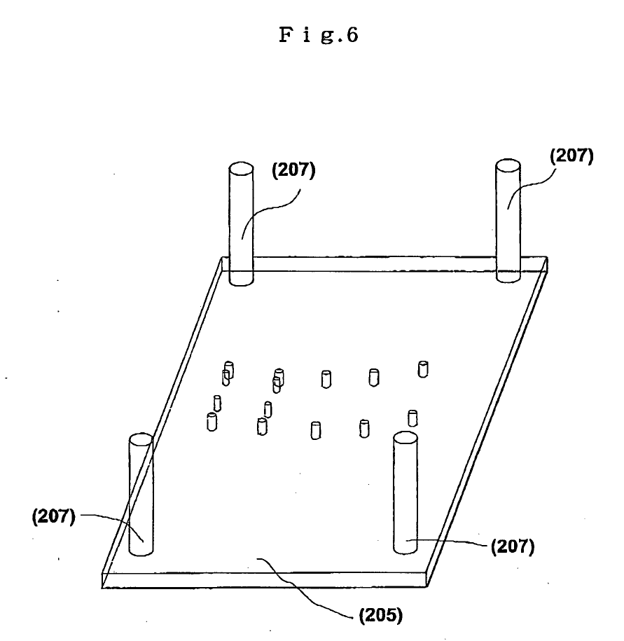

FIGS. 2 to 6 show each of the lamination substrates constituting the blood analysis apparatus, wherein FIG. 2 represents the uppermost layer of the substrate 201, FIG. 3 represents the second layer of the substrate 202, FIG. 4 represents the third layer of the substrate 203, FIG. 5 represents the fourth layer of the substrate 204 and FIG. 6 represents the lowest layer of the substrate 205. Each of the substrates is made from polycarbonate having a thickness of 0.5 mm, and the flow channels or holes are formed by injection molding. Of course, it is also manufactured by molding a pattern such as SU-8 which is manufactured by lithography, on a polymer substrate such as a PET (polyethylene terephthalate) plate, so the manufacturing method has no feature. Into the holes 206 which are installed on each of the layer of the substrate in FIGS. 2 to 6 are inserted the cylinder 207, whereby the fourth layer of the substrate 204 is connected onto the lowest layer of the substrate 205, the third layer of the substrate 203 onto the fourth layer of the substrate 204, the second layer of the substrate 202 onto the third layer of the substrate 203, and the uppermost layer of the substrate 201 onto the second layer of the substrate 202, and each of the substrates adheres by an adhesive, etc. As results, locations of the flow channels which are installed in each of the substrates are determined, which makes it possible flow of a solution between each of the substrates. Then, the second layer of the substrate 202 will be explained. 208 shows a no-pain needle which is a stainless tube having an outer diameter of 100 μm and an internal diameter of 50 μm, and of which the tip is polished from three planes by 10 degrees. Since the inner wall is polished to ultra-smoothing, it is possible to collect blood automatically by the blood pressure by injecting it into the vein. In addition, 209 is a stainless tube having an outer diameter of 250 μm, which adheres to 208. 210 is a glass tube having an outer diameter of 1.8 mm and an internal diameter of 1 mm, which adheres to 209. The blood collected from the no-pain needle 208 is reserved in the glass tube 210. 211 is a guiding flow channel for delivery of the blood, which is processed on the substrate 202. If the centrifugal force is applied for the center of the first rotation axis 212 of the second substrate 202, the blood is introduced into plural blood reservoir grooves 213. If the centrifuge further continues, the blood cells are isolated to the outer side, and the plasma is isolated to the inner side from the view of the first rotation axis 212. Then, if the centrifugal force is applied for the center of the second rotation axis 214, the supernatant plasma is introduced into a plasma reservoir for weighing 216 via a guiding groove for delivery of the blood 215. At this time, a hole for introduction of the air 217 and a guiding groove therefor 218 are installed to make the introduction possible. The hole for introduction of the air 217 is directly connected to a hole for introduction of the air 219 on the uppermost layer of the substrate 201. Extra plasma after filling the plasma reservoir for weighing 216 is disposed to a reservoir for waste of extra plasma 221 via a guiding flow channel for extra plasma 220. Furthermore, if the centrifugal force of 1000 G or more is applied for the center of the second rotation axis 214, the plasma reservoir for weighing 216 is introduced into plural channels which are installed on the substrate 202, via a capillary valve 222.

The capillary valve described above is described in Nam-Trung Nguyen and Steven T. Wereley, “Fundamentals and Applications of Microfludics”, page 315, published by Artech House (Boston, London), 2002. Explaining briefly, under presence of a solution in a tubule (capillary), there is a rotation axis on the extension of one end in the axial direction of the tubule, and the solution is reserved on the other end of the tubule by the surface tension, and if the tubule is rotated about its rotation axis, the solution is not discharged from the other end of the tubule with small centrifugal force, but if the centrifugal force increases, it beats the surface tension lastly, and the solution is discharged. Therefore, the magnitude of the centrifugal force has the action of a valve, which makes it called as a capillary valve. The minimum rotation number fm at which the solution is discharged from the tubule, is present between the tubule radius R₁ on the side of the rotation axis, and the radius R₂ on the side of the discharge, and they are in a relation as below.

fm²≧γ cos θ/R·ρ·π²·(R₂−R₁)(R₂+R₁)

wherein θ represents contact angle of the solution to the tubule when the solution is discharged from the tubule, γ represents the surface tension, R represents the radius of the tubule, and ρ represents the density of the solution.

γ is 72×10⁻³ [N/m] in water of 25° C., the contact angle θ with water is 80 degrees when PET is used as a substrate, and ρ of water is 1×10⁻³ [kg/m³]. With the use of these values, if R₂ is 5 cm, the length of the capillary valve, (R₂−R₁) in sum is 0.5 cm and the diameter (2 R) is about 100 μm, the plasma flows into the flow channel for the dry chemistry reagent from the plasma reservoir for weighing when fm is about 1000 rotation/second or more. The gravity acceleration is 60 G or more at this time.

There are at least two kinds of reaction systems in the dry chemistry. One of them is measurement of creatinine or urea nitrogen, and the other is measurement of glucose, γ-GTP, GOT, GPT or total cholesterol. The former measurement uses two steps of reactions, i.e., first, generation of ammonia (NH₃) gas with reaction with each enzyme of creatinine deamylase in the case of creatinine or urease in the case of urea nitrogen, and reaction of NH₃ gas to each of bromcresol green and bromphenol blue for color development. On the other hand, the other test substance is detected with one-step reaction of the enzyme corresponding to each of the reactions and the gel containing a colorant reagent. Herein, 223 represents a flow channel for detection of NH₃, and 224 represents a flow channel for detection of other assay items than NH₃, each of which is filled with the gel described above.

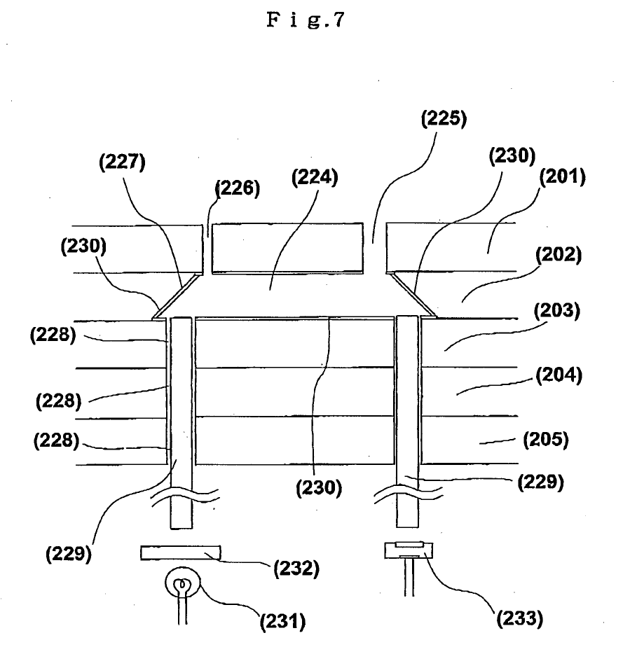

FIG. 7 shows the structure of the lamination substrates wherein a flow channel is installed for other assay items than NH₃. The holes 225 and 226 on the uppermost layer of the substrate 201 are openings for introduction and discharge of the gel, respectively, and the diameter of the opening for introduction is more than that of the opening for discharge. Introduction of the gel containing the reagent is carried out with a micro-disc sensor. The openings for introduction and discharge are holes for injection of the gel into the flow channels of two different reaction systems 223 and 224 and for discharge of extra gel after filling up the inside, and for discharge of swollen gel to the outside since the gel swells to two to five folds if the plasma is introduced into the gel by the centrifugal force, though it depends on the kind of the gel. In addition, a declined wall 227 of 45 degrees is formed on both ends of the longitudinal direction of the flow channel 224. Its inner wall is spark-deposited with an aluminum film, and a through-hole 228 is installed on the third layer of the substrate 203, the fourth layer of the substrate 204 and the lowest layer of the substrate 205, into which a fiber 229 is inserted, of which the edge stays below the declined wall of 45 degrees. A method of the sputter deposition of the aluminum film 230 on the inner wall of the flow channel 224, comprises bringing the uppermost layer of the substrate 201 into contact with the second layer of the substrate 202, sputter depositing aluminum from the back side of the second layer of the substrate 202, and removing the aluminum film in other part than the flow channel by CMP (Chemical Mechanical Polishing). Next, the method comprises removing the uppermost layer of the substrate 201 from the second layer of the substrate 202, bringing the second layer of the substrate 202 into contact with the third layer of the substrate 203, spark-depositing aluminum from the upside of the uppermost layer of the substrate 201, and removing the aluminum film in other part than the flow channel by CMP. In addition, any other metal material may be used besides aluminum if it is a material which reflects light within the flow channel in good efficiency without runaway of the light to the outside. The light having the wavelength which is selected through the bandpass filter 232 from the halogen lamp or tungsten lamp 231 is introduced into the fiber 229, and its attenuation degree is measured by the optical receiver 233.

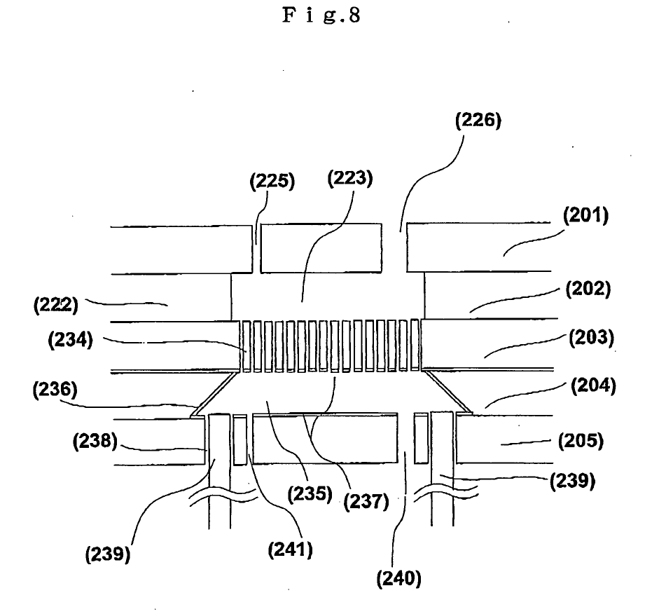

FIG. 8 shows the structure of the flow channel of multiple layers including the flow channel for detection of ammonia gas 223. Plural holes 234 are installed which have the diameter of about 5 μm to 50 μm, and functions to pass ammonia gas, on the third layer of the substrate 203. The area where these plural holes are installed is the same as that of the flow channel 223 which receives a reagent for generation of ammonia gas and is installed on the second layer of the substrate 202. This region of the holes 234 also serves as a layer for development of the dry chemistry. FIG. 4 is an airplane view thereof. For the manufacturing method for the hole, in the case of large diameter, it can be formed with stainless or mesh made of polymer or injection molding, and in the case of small diameter, it can be formed with deep etching on a silicone substrate. NH₃ gas which has passed through the multiple holes 234, is introduced into the flow channel described in FIG. 8, which is formed on the fourth layer of the substrate 204 and the lowest layer of the substrate 205. A method for coating the aluminum film onto the inner wall of the flow channel is similar to those described above. A gel containing a reagent which reacts to ammonia gas and give coloration is introduced into a flow channel 235. Herein, 236 represents a declined wall of 45 degrees, 237 represents an aluminum film, 238 represents a through-hole for the fiber, 239 represents a fiber, and 240 and 241 represent holes for injection and discharge of the gel containing the reagent which reacts to ammonia gas and give coloration. Optical parts such as a lamp, bandpass filter and optical receiver are not shown and skipped.

### Second Embodiment

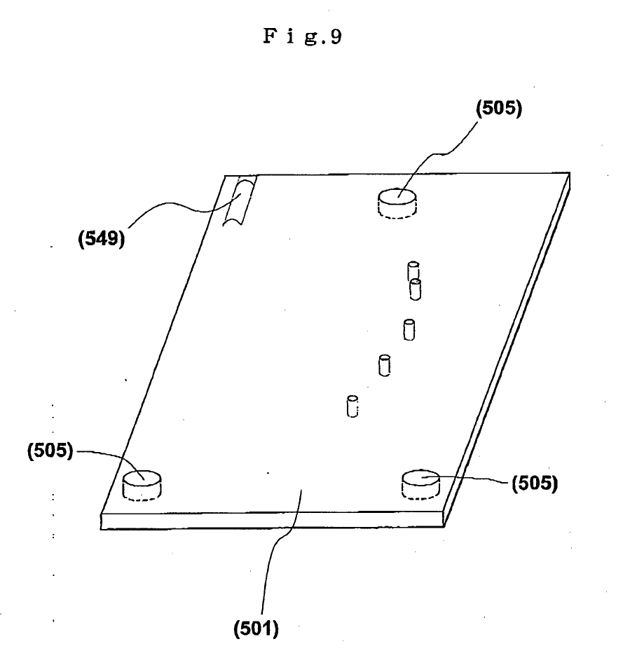

FIGS. 9 to 12 show each of the lamination substrates constituting the blood analysis apparatus, wherein multiple colorimetric sensors and multiple electrochemical sensors are integrated on one substrate. pH and the ion concentration of Na⁺⁺, K⁺, Cl⁻, Ca⁺⁺, etc., which are basically necessary, generally need to be measured in diagnosis. pH is measured by amperometry, and each concentration of Na⁺⁺, K⁺, Cl⁻ and Ca⁺⁺ is measured by potentiometry, which are electrochemical methods. Accordingly, it is desired that both of the colorimetric sensor and the electrochemical sensor are integrated on one substrate, and measurement is made by one blood collection. The calorimetric sensor for NH₃ detection is not loaded into this group of the colorimetric sensors due to complicity of constitution, but in the case of NH₃ detection, a layer for generation of NH₃ gas is added similarly to those in FIGS. 2 to 6.

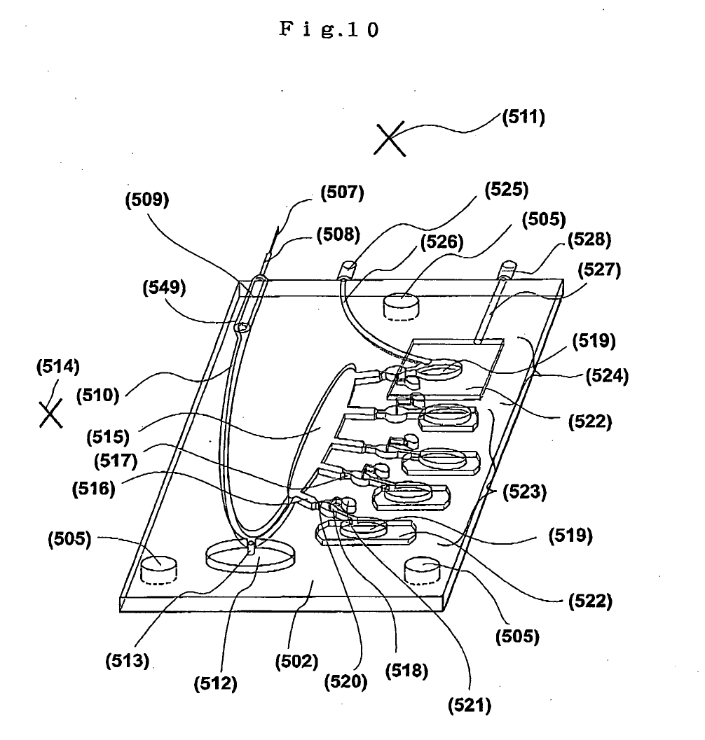

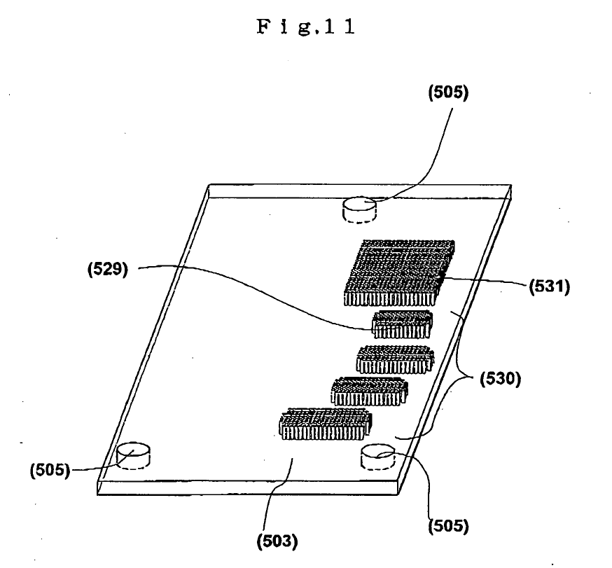

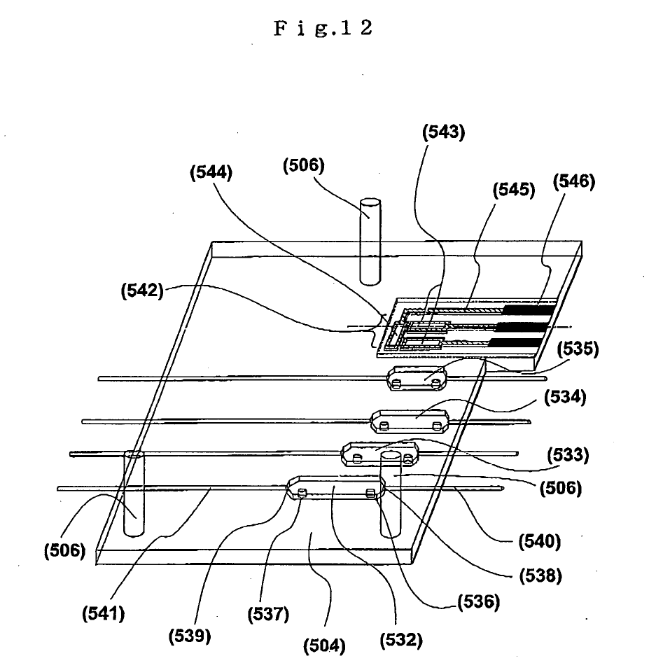

It is composed of 4 pieces of substrates, which are the uppermost layer 501 represented by FIG. 9, the second layer 502 represented by FIG. 10, the third layer 503 represented by FIG. 11 and the lowest layer 504 represented by FIG. 12. Similarly to those in FIGS. 2 to 6, the third layer 503 is connected onto the lowest layer 504, the second layer 502 onto the third layer 503, and the uppermost layer 501 onto the second layer 502, by insertion of the cylinder 506 into the hole 505 in each of the lamination substrates in FIGS. 9 to 12, and each of the substrates adheres by an adhesive, etc. Further, on the second layer of the substrate 502, a no-pain needle 507 is connected to a glass tube 509 via a stainless tube 508, and the blood is collected in the glass tube 509 similarly to those in FIG. 3. The blood is introduced into an approximately U-shaped tube 510 which is opened by the centrifugal force for the center of the fist rotation axis 511, and if the centrifuge further continues, the blood cell fraction is isolated to the outer side, and the plasma fraction is isolated to the inner side from the view of the first rotation axis 511. The blood cell fraction is reserved in a blood cell reservoir 512. 513 is a column to prevent return of the blood cell to the U-shaped tube. Then, if the centrifugal force is applied for the center of the second rotation axis 514, the supernatant plasma is once introduced into a plasma reservoir 515. Furthermore, if the rotation continues, it is introduced into a plasma reservoir for weighing plasma 518 and a flow channel for introduction of the plasma 519 through a capillary valve 517 by the centrifugal force with the gravity acceleration of 60 G or more via a guiding flow channel for delivery of the plasma 516. At this time, the extra plasma after weighing is wasted to a waste plasma reservoir 521 on the lateral wall of the plasma reservoir for weighing plasma 518 via a guiding flow channel for delivery of waste plasma 520. A layer for developing plasma 522 is installed right below the flow channel for introduction of the plasma 519, and the plasma is once expanded on a flat plane. Herein, 523 represents a sensor region of the dry chemistry-colorimetric method, and 524 represents a sensor region of the electrochemical sensor method. The electrochemical sensor needs calibration before measurement, and 525 represents an opening for introduction of a calibrated solution, 526 represents a guiding flow channel for introduction of a calibrated solution, 527 represents a guiding flow channel for waste solution after calibration and 528 represents a waste solution opening of the waste solution after calibration.

As shown in FIG. 11, a region 529 wherein multiple holes having the diameter of about 5 μm to 50 μm are installed, is formed right below a layer for developing the plasma 522 similarly to the third layer 503 in FIG. 4, and the area and the location of the region are in conformity with those of the layer for developing the plasma 522. Herein, 530 represents a sensor region of the dry chemistry-colorimetric method, and 531 represents a sensor region of the electrochemical sensor method.

As shown in FIG. 12, channels for colorimetric measurement wherein the dry chemistry reagent is introduced 532, 533, 534 and 535, which have the same area, are joined at the same location right below the layer for developing the plasma 522 on the lowest layer 504. The inner wall of these channels is coated with a metal such as aluminum which reflects light according to the method described in FIG. 7. 536 and 537 are openings for injection and discharge, respectively of the dry chemistry reagent. An opening for incidence 538 of the measurement light is installed on one end, and an opening for discharge 539 after propagation of the flow channel is installed on the other end of the lateral wall in the longitudinal direction of the flow channel for colorimetric measurement wherein the dry chemistry reagent is introduced 531. Fibers 540 and 541 are connected into each of the openings, serving incidence of tile measurement light and emission to the received light. Both of the fibers are installed on the substrate 504 in conformity with the center axis in the longitudinal direction of the flow channels for colorimetric measurement wherein the dry chemistry reagent is introduced 532 to 535. Attenuation degree of the introduced light by color development of the reagent is detected by a photo detector by optical diode, etc.

The plasma from the layer for developing the plasma 522 is introduced into the flow channels for the dry chemistry reagent 532 to 535 and a region of electrochemical sensor 542 through a region 529 which is right below it and where multiple holes are formed. The centrifugal force used in the introduction is in the perpendicular direction with the hole, and the centrifugal force has no action in the vertical direction. However, the end of the flow channel for plasma reservoir in the direction where the centrifugal force is applied to, serves as a wall, and the flow channel for plasma reservoir is full of the plasma, which leads the plasma received pressure by the centrifugal force to be introduced into the hole below. Like this, it is possible to introduce the plasma into the flow channels for dry chemistry reagent 532 to 535 and the region of electrochemical sensor 542 only with the use of the centrifugal force.

The reason will be described below why the lengths of the flow channels 532 to 535 in the longitudinal direction are shown to be different. When the reagent is dispersed in the gel, a gel is needed which is suitable for each reagent in order to make the reagent compatible with the gel, but various gels have different swelling degree when they react to the plasma, and the gel usually swells to 2 to about 5 folds. Herein, the flow channels for the dry chemistry reagent 532 to 535 should be designed to have optimal length to allow the weighed, supplied plasma to react to the gel in a maximum. Openings for injection and discharge of the gel containing the reagent 536 and 537 also serve to discharge the gel which has swollen over the volume of the flow channel to the outside. However, the attenuation degree can be measured by introducing a certain light in the total swollen volume, and measurement values having a large S/N ratio are obtained. Reversely, the measurement may have trouble if the attenuation degree is too high, and the area occupied but the flow channel increases, which leads to increase of the substrate area.

On the other hand, the flow channel for detection of NH₃ 223 and the flow channel for detection of other assay items than NH₃ 224 are shown to have the same area and volume in FIG. 3. For the reagent which has largely swollen, the introduced reagent is discharged from the discharge hole, which leads to small attenuation degree of the measurement light and measurement values having a low S/N ratio, but it is possible to measure the test component concentrations of multiple items even with the now channel of a constant volume, by knowing in advance the concentration of the test component of the user and preparing a measurable dry chemistry reagent. The both of the inventions are used in combination in the real substrate.

542 is a region of an electrochemical sensor. 543 is an Ag/AgCl electrode, onto which a sensor film containing ionophore for Na⁺ or K⁺ ion is coated. 544 is a KCl-saturated Ag/AgCl reference electrode, and these electrodes are installed on silver carbon wiring 545, and 546 is an outside electrode for taking the signal out.

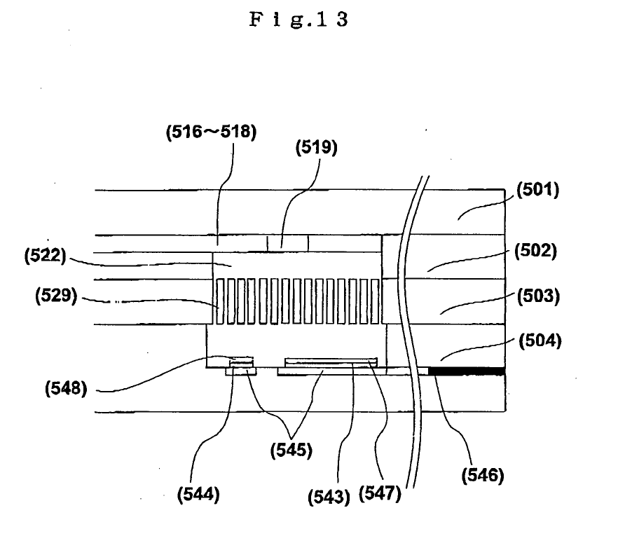

FIG. 13 is a view showing the section of the structure of the electrochemical sensor region 542, which is shown as dashed oblique line in FIG. 12.

The electrochemical sensor is first calibrated before introduction to the plasma. Then, the plasma passes through a capillary valve 517 via a guiding flow channel for delivery of the plasma 516 which is installed on the second layer 502 by the centrifugal force with the gravity acceleration of 60 G or more, and it is introduced into a plasma reservoir for weighing plasma 518, a guiding flow channel for introduction of the plasma 519 and a layer for developing of the plasma 522, and then introduced into an electrochemical sensor region 542 through a region 529 where multiple holes are formed. Herein, 547 is an ionophore film, and 548 is a film for prevention that KCl of the KCl-saturated Ag/AgCl reference electrode dissolves and is lost in the electrolytic solution.

### EXAMPLES

### Example 1

Measurement of Glucose

An example, in which glucose measurement was carried out using the blood analysis apparatus based on FIGS. 2 to 6, is shown. The principle of measurement is that if a colorant reagent acts on a sample, glucose in the sample is converted from α form to β from rapidly by mutarotase contained in the colorant reagent. β-D-glucose is oxidized by the action of glucose oxidase (GOD), and hydroperoxide are produced at the same time. Produced hydroperoxide is oxidatively condensed quantatively with phenol and 4-aminoantipyrine in the colorant regent by the action of coexisting peroxidase (POD) to give a red pigment (505 nm). Absorbance of this red pigment is measured to determine the glucose concentration in the sample.

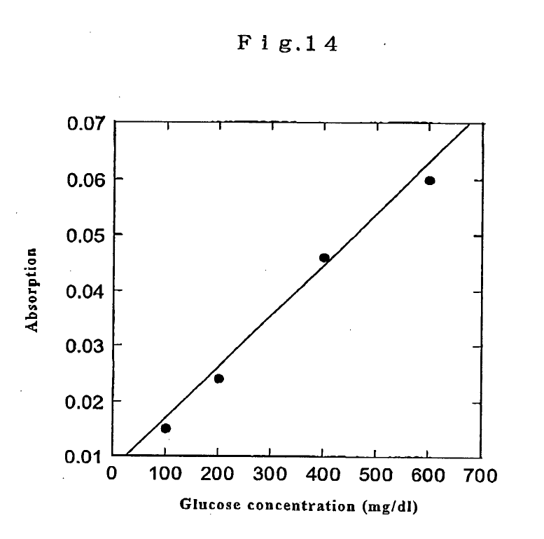

The dry chemistry reagent was prepared by mixing powders of (1) 1.8 U of glucose oxidase derived from Aspergillus niger, black fungus, (2) 0.03 mg (0.188 μmol) of 1.7-dihydroxynaphthalene, (3) 0.1 mg (0.492 μmol) of 4-aminoantipyrine, (4) 0.13 U of peroxidase and (5) 0.065 U of mutarotase (derived from pig kidney), and dissolving them in a phosphate buffer solution. Then, all of this dissolved solution was contained in gelatin to form a gelatin gel. This was injected into a flow channel for calorimetric measurement wherein a dry chemistry reagent is introduced. The flow channel for colorimetric measurement had a sectional area of 0.4 mm×0.4 mm and a length of 1 mm. Into this channel, the gel described above was injected. Further, 0.2 μL of glucose standard solution was changed from 0 mg/dl to 600 mg/dl in glucose concentration. 505 nm of light was irradiated into this channel and the obtained absorbance was shown in FIG. 14. This result shows that the detection was good.

### Example 2

Measurement of Urea Nitrogen

An example, in which urea nitrogen measurement was carried out using the blood analysis apparatus based on FIGS. 7 to 12, is shown.

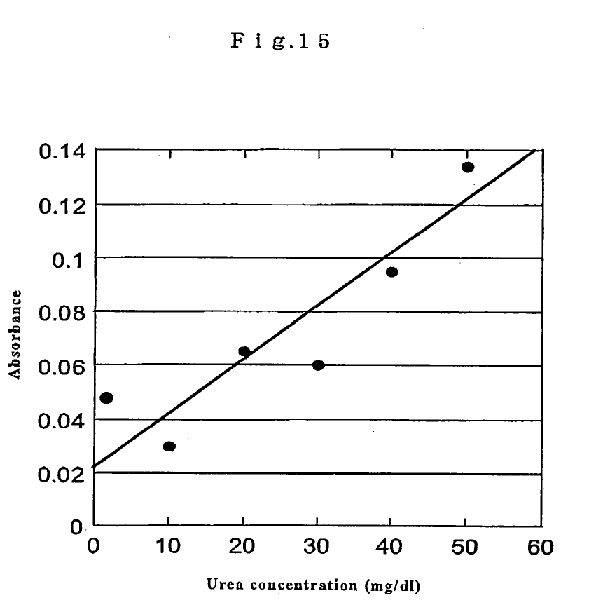

Urea nitrogen (H₂NCONH₂) is decomposed into NH₃ and CO₂ with the action of urease in presence of water, and this NH₃ reacts to bromcresol green to give blue pigment (620 nm). Measurement of urea was performed as follows. A gel, wherein 2 ml of a urease solution was contained in 10 mg of water-absorbing polymer comprising starch-acrylonitrile, was injected into a flow channel for NH₃ generation having a sectional area of 0.4 mm×0.4 mm and a length of 1 mm. A colorant reagent was prepared by dissolving 20 mL of isopropyl alcohol and 1.28 mg of PVB (polyvinylbutyral) of 5% weight ratio together in a 3% solution of bromcresol green (BCG) (weight ratio) dissolved in 2 ml of ethanol, and injected into a flow channel for color development. The substrate where multiple holes were formed had a thickness of 1.2 mm. FIG. 15 shows absorbance when 620 nm of light was irradiated into a urea standard solution which was prepared from 10 mg to 50 mg. This result shows that the detection was good.

### INDUSTRIAL APPLICABILITY

As shown above, the blood analysis apparatus of the present invention can accomplish a blood analysis apparatus for multiple items which is cheap and simple, and makes it possible to measure test components for multiple items from the plasma of very small amount of the blood, by performing a series of operations, i.e., introduction of the whole blood into a substrate, separation of the blood cells and the plasma, weighing of the plasma and development to a dry chemistry reagent, by the centrifugal force and without the use of a pump, and measuring optical attenuation in the longitudinal direction of the flow channel into which the dry chemistry reagent has been introduced.

The blood analysis apparatus of the present invention can accomplish a blood analysis apparatus which makes it possible to measure ammonia gas in high sensitivity in measurement of ammonia gas due to the laminated and isolated structure of a layer for generation of ammonia gas, a layer for development of ammonia gas and a layer for color development by reaction with ammonia gas. As results, an electrochemical sensor including ammonia gas measurement can be combined in addition to the colorimetric sensor of dry chemistry, which allows measurement of most usual blood analysis items, and leads to accomplish diagnosis at home by use of no-pain needle, not only at bedside.

1. A blood analysis apparatus for a test component in the plasma by a laminated substrate which has an opening for introduction of the blood, a guiding flow channel which is connected thereto for delivery of the whole blood, and a flow channel for separation of the blood cells and the plasma and a guiding flow channel for the plasma, and which is provided with each means of introducing a whole blood sample from the opening for introduction of blood with the use of the centrifugal force, performing separation of the blood cells and the plasma, delivering the plasma by the guiding flow channel, introducing it into a flow channel which has received a dry chemistry reagent to allow the test component in the plasma to react to the dry chemistry reagent, and introducing a certain light for measurement to thereby measure the change of permeation degree by an optical receiver.

2. The blood analysis apparatus according to claim 1, wherein a blood-collection needle and a blood reservoir where the blood has been collected are connected nearly linearly into the blood introduction opening, and further the flow channel for separation of the blood cells and the plasma is connected into the blood introduction opening via the guiding flow channel for delivery of the whole blood, the whole blood is delivered into the flow channel for separation of the blood cells and the plasma via the guiding flow channel for the blood from the blood introduction opening by rotation about the first rotation axis upward from the view of the blood introduction opening from the flow channel for separation of the blood cells and the plasma, and it is isolated into the blood cell fraction and the plasma fraction.

3. The blood analysis apparatus according to claim 1, wherein the guiding flow channel for delivery of the whole blood and the flow channel for separation of the blood cells and the plasma are installed downward from the view of the blood introduction opening, plural groove flow channels are installed in the flow channel for separation of the blood cells and the plasma in an approximately arc shape about the blood introduction opening and further toward the blood introduction opening, the lower side from the view of the blood introduction opening of the groove flow charm the whole blood is rotated about the first rotation axis, whereby the blood cell fraction is received in the lower side of the groove flow channel, and the plasma fraction is received in the upper side of the groove flow channel.

4. The blood analysis apparatus according to claim 1, wherein the now channel for separation of the blood cells and the plasma is formed in approximately U-shaped flow channel, one end of the U-shaped flow channel is connected to the guiding flow channel for delivery of the blood which is connected to the whole blood introduction opening; the whole blood is introduced into the U-shaped flow channel by rotation about the first rotation axis, which is installed upward on the substrate from the view of the blood introduction opening from the lower end of the U-shaped flow channel, and a part receiving the blood cell fraction is installed in the curved part of the lower end of the U-shaped flow channel, and a part receiving the plasma fraction is installed hi the upper part thereof.

5. The blood analysis apparatus according to claim 3, wherein each of the other ends of plural groove flow channels has a guiding flow channel for the plasma, a plasma reservoir for weighing the plasma, a capillary valve and each flow channel for introducing a dry chemistry reagent, which are connected in a series in this order, rotation is made about the second rotation axis which is installed in the outer lower side of plural groove flow channels, and the plasma from the part receiving the plasma fraction is introduced into each of channels for introduction of a dry chemistry reagent via each of the plasma guiding flow channels, each of the plasma reservoirs for weighing the plasma and each of the capillary valves by the centrifugal force.

6. The blood analysis apparatus according to claim 4, wherein a laminated substrate is formed by installing a guiding flow channel for delivery of the blood, which is branched in plural number in the flow channel of the other end of the U-shaped flow channel, and which has a blood reservoir, a plasma reservoir for weighing, a capillary valve and a plasma reservoir for weighing the plasma and a flow channel for introduction of the plasma, which are connected to in a series in this order, and further installing a layer for development of the plasma right below the flow channel for introduction of the plasma, and joining a substrate, which is joined in conformity in the vertical direction right below the layer for development of the plasma, and which has a region of the same area as that of the layer for development of the plasma where plural holes are installed, and a flow channel for calorimetric measurement wherein a dry chemistry reagent is introduced, which is installed right below the porous substrate, and has the same area as that of the layer for development of the plasma, in this order, and in a direction about perpendicular to the U-shaped flow channel, the laminated substrate is rotated about the second rotation axis located in the outside of the substrate from the view from the flow channel, whereby the plasma in the U-shaped flow channel is introduced into the flow channel for colorimetric measurement wherein a dry chemistry reagent is introduced by the centrifugal force.

7. The blood analysis apparatus according to claim 5, wherein a guiding flow channel for delivery of the waste plasma and a waste plasma reservoir which is connected thereto, are installed on the lateral wall of the plasma reservoir in the plasma reservoir for weighing the plasma.

8. The blood analysis apparatus according to claim 5, wherein an opening for injection and an opening for discharge of the dry chemistry reagent are installed in the flow channel of gathering the dry chemistry reagent.

9. The blood analysis apparatus according to claim 6, wherein in the flow channel for colorimetric measurement of the dry chemistry reagent, the both ends of the flow channels in the longitudinal direction have walls installed in 45 degree, and all of the inside is coated with metal, and a fiber for optical introduction and emission is introduced around the wall of 45 degree vertically from the lower part or upper part of the wall substrate of 45 degree.

10. The blood analysis apparatus according to claim 6, wherein in the flow channel for calorimetric measurement of the dry chemistry reagent, the inside is coated with metal, and a fiber for optical introduction and emission is introduced from both of the directions in conformity with the flow channel axis on the wall of the both ends of the flow channels in the longitudinal direction.

11. The blood analysis apparatus according to claim 6, wherein the flow channel for colorimetric measurement of the dry chemistry reagent has a volume to which a gel containing the dry chemistry reagent swells in reaction with the plasma.

12. The blood analysis apparatus according to claim 6, wherein the flow channel for calorimetric measurement of the dry chemistry reagent has the constant volume.

13. The blood analysis apparatus according to claim 6, wherein in the flow channel for colorimetric measurement of the dry chemistry reagent, a substrate of the flow channel of the dry chemistry reagent for generation of ammonia gas, a substrate wherein a number of holes are installed and a flow channel for colorimetric measurement wherein a coloration material for generation and detection of ammonia gas is introduced, are laminated in this order in conformity in the vertical direction, and further wherein the flow channel of the dry chemistry reagent for generation of ammonia gas, the region where a number of holes are installed and the flow channel for calorimetric measurement wherein a coloration material for generation and detection of ammonia gas is introduced have the same area, and further a flow channel for detection of other items are installed on the same substrate with the flow channel for generation of ammonia gas.

14. The blood analysis apparatus according to claim 1, wherein a colorimetric sensor with the use of a dry chemistry reagent and an electrochemical sensor are installed in the blood analysis apparatus which is constituted by the lamination substrates.

15. The blood analysis apparatus according to claim 2, wherein the guiding flow channel for delivery of the whole blood and the flow channel for separation of the blood cells and the plasma are installed downward from the view of the blood introduction opening, plural groove flow channels are installed in the flow channel for separation of the blood cells and the plasma in an approximately arc shape about the blood introduction opening and further toward the blood introduction opening, the lower side from the view of the blood introduction opening of the groove flow charm the whole blood is rotated about the first rotation axis, whereby the blood cell fraction is received in the lower side of the groove flow channel, and the plasma fraction is received in the upper side of the groove flow channel.

16. The blood analysis apparatus according to claim 2, wherein the now channel for separation of the blood cells and the plasma is formed in approximately U-shaped flow channel, one end of the U-shaped flow channel is connected to the guiding flow channel for delivery of the blood which is connected to the whole blood introduction opening; the whole blood is introduced into the U-shaped flow channel by rotation about the first rotation axis, which is installed upward on the substrate from the view of the blood introduction opening from the lower end of the U-shaped flow channel, and a part receiving the blood cell fraction is installed in the curved part of the lower end of the U-shaped flow channel, and a part receiving the plasma fraction is installed in the upper part thereof.

17. The blood analysis apparatus according to claim 6, wherein a guiding flow channel for delivery of the waste plasma and a waste plasma reservoir which is connected thereto, are installed on the lateral wall of the plasma reservoir in the plasma reservoir for weighing the plasma.

18. The blood analysis apparatus according to claim 6, wherein an opening for injection and an opening for discharge of the dry chemistry reagent are installed in the flow channel of gathering the dry chemistry reagent.
